(19) United States
(12) Patent Application Publication
(10) Pub. No.: US 2019/0057189 A1
(43) Pub. Date: Feb. 21, 2019

(54) Alert and Response Integration System, Device, and Process

(71) Applicant: Innovative World Solutions, LLC, Asburn, VA (US)

(72) Inventors: Steven James Frederickson, Ashburn, VA (US); Cynthia Lee Chambers, Ashburn, VA (US)

(21) Appl. No.: 15/679,705

(22) Filed: Aug. 17, 2017

(51) Int. Cl. G06F 19/00; G08B 21/04; G08B 25/01; A61B 5/00; A61B 5/11; A61B 5/0205

(52) U.S. Cl. CPC G06F 19/3418; G08B 21/0453; G08B 21/043; G08B 21/0469; A61B 5/024; A61B 5/746; A61B 5/1112; A61B 5/0022; A61B 5/0205; G08B 25/016

(57) ABSTRACT

The disclosure is directed to an alert and response integration system device. The device includes at least one physiological data sensor configured to sense physiological data of a user; at least one location determination device configured to determine a location of the user; a memory configured to store a monitoring application; and a processor configured to execute a monitoring application and obtain the physiological data from the at least one physiological data sensor and obtain the location of the user from the at least one location determination device. The device further includes a transceiver configured to transmit an alert over a wireless network to a monitoring device and an attachment mechanism associated with the housing.

### BACKGROUND OF THE DISCLOSURE

### 1. Field of the Disclosure

This disclosure relates generally to an alert and response integration system, device, and process. More specifically, the disclosure relates generally to an alert and response integration system, device, and process that improves supervision and oversight of children, adults, pets, and the like.

### 2. Related Art

The leading causes of death in the United States are heart disease, cancer, chronic lower respiratory disease, accidents, stroke, Alzheimer's disease, diabetes, and influenza and pneumonia. The leading causes of death in the home in the United States are falls, poisoning, fire, suffocation, choking, and drowning. Moreover, the issues in the home are more likely to happen to children and the elderly. Ensuring the oversight and/or well-being of children, adults, pets and the like by individuals in charge of their well-being is of paramount importance. For example, parents, guardians, teachers, caregivers, and the like charged with the well-being of children need to ensure that children are safe and protected. Likewise, adult children, parents, guardians, caregivers, and the like charged with the well-being of elders or individuals with limited capacity need to ensure that these individuals are safe and protected. Similarly, pet owners, caregivers, and the like charged with the well-being of pets need to ensure that pets are safe and protected. Finally, law enforcement officers, airport security personnel, and the like charged with the oversight of individuals of concern need to ensure that these individuals of concern are acting appropriately.

A number of approaches have been made to ensuring the supervision, oversight and/or well-being of children, adults, and pets by individuals. However, these approaches have a limited capability and accordingly limited usefulness.

Accordingly, there is a need for an alert and response integration system, device, and process that provides greater capability, usefulness, and the like to protect, benefit, and ensure the well-being, supervision, and/or oversight of children, adults, pets, and/or society as a whole.

### SUMMARY OF THE DISCLOSURE

The foregoing needs are met, to a great extent, by the disclosure, with an alert and response integration system, device, and process that provides greater capability, usefulness, and the like to protect, benefit, and ensure the supervision, oversight, and/or well-being of children, adults, pets, and/or society as a whole.

In one or more aspects an alert and response integration system device includes at least one physiological data sensor configured to sense physiological data of a user; at least one location determination device configured to determine a location of the user; a memory configured to store a monitoring application; a processor configured to execute the monitoring application and obtain the physiological data from the at least one physiological data sensor and obtain the location of the user from the at least one location determination device; the processor further configured to generate an alert if the physiological data exceeds a predetermined threshold; the processor further configured to generate the alert if the location of the user is a particular location; a transceiver configured to transmit the alert over a wireless network to a monitoring device; a housing configured to house the at least one physiological data sensor, the at least one location determination device, the memory, the processor, and the transceiver; and an attachment mechanism associated with the housing, the attachment mechanism being configured to securely affix the housing to the user with a locking mechanism, wherein the locking mechanism is configured to be unlocked by a caregiver.

In one or more aspects an alert and response integration process includes sensing physiological data of a user with at least one physiological data sensor; determining a location of the user with at least one location determination device; storing a monitoring application in a memory; executing the monitoring application with a processor and the processor obtaining the physiological data from the at least one physiological data sensor and the processor obtaining the location of the user from the at least one location determination device; generating with the processor an alert if the physiological data exceeds a predetermined threshold; generating with the processor the alert if the location of the user is a particular location; transmitting the alert over a wireless network with a transceiver to a monitoring device; providing a housing configured to house the at least one physiological data sensor, the at least one location determination device, the memory, the processor, and the transceiver; and providing an attachment mechanism associated with the housing, the attachment mechanism being configured to securely affix the housing to the user with a locking mechanism, wherein the locking mechanism is configured to be unlocked by a caregiver.

There has thus been outlined, rather broadly, certain aspects of the disclosure in order that the detailed description thereof herein may be better understood, and in order that the present contribution to the art may be better appreciated. There are, of course, additional aspects of the disclosure that will be described below and which will form the subject matter of the claims appended hereto.

In this respect, before explaining at least one aspect of the disclosure in detail, it is to be understood that the disclosure is not limited in its application to the details of construction and to the arrangements of the components set forth in the following description or illustrated in the drawings. The disclosure is capable of aspects in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the disclosure. It is important, therefore, that the claims be regarded as including such equivalent constructions insofar as they do not depart from the spirit and scope of the disclosure.

### BRIEF DESCRIPTION OF THE DRAWINGS

FIG. 1 illustrates an exemplary alert and response integration system device with associated components, in accordance with aspects of the present disclosure.

FIG. 2 schematically illustrates the details of an exemplary alert and response integration system device in accordance with aspects of the disclosure.

FIG. 3 illustrates the details of an exemplary alert and response integration system device in accordance with aspects of the disclosure.

FIG. 4 illustrates a process of operating the alert and response integration system device in accordance with aspects of the disclosure.

FIG. 5 illustrates an exemplary implementation of an alert and response integration system device with associated components, in accordance with aspects of the disclosure.

FIG. 6 illustrates an exemplary implementation of an alert and response integration system device with associated components, in accordance with aspects of the disclosure.

FIG. 7 illustrates an exemplary implementation of an alert and response integration system device with associated components, in accordance with aspects of the disclosure.

FIG. 8 illustrates an exemplary implementation of an alert and response integration system device with associated components, in accordance with aspects of the disclosure.

FIG. 9 illustrates an exemplary implementation of an alert and response integration system device with associated components, in accordance with aspects of the disclosure.

### DETAILED DESCRIPTION

Reference in this specification to an alert and response integration system (ARIS) device is intended to encompass devices such as wireless devices, internet-enabled devices, smartphones, mobile phones, tablet computers, MP3 players, personal digital assistants (PDAs), and the like. For example, the ARIS device is intended to encompass any compatible mobile technology computing device that connects to a wireless communication network, such as mobile phones, mobile equipment, mobile stations, user equipment, cellular phones, smartphones, handsets or the like (e.g., Apple iPhone, iPad, Google Android based devices, BlackBerry based devices, other types of PDAs or smartphones), wireless dongles, or other mobile computing devices. The term “alert and response integration system device” may be interchangeably used and referred to herein as “wireless device,” “wireless handset,” “handset,” “mobile device,” “device,” “mobile phones,” “mobile equipment,” “mobile station,” “user equipment,” “cellular phone,” “smartphones,” “phone” or the like.

The ARIS device may connect to a “wireless network” or “network” and is intended to encompass any type of wireless network to obtain wireless services through the use of an alert and response integration system, such as the Global System for Mobile Communication (GSM) network, Code-Division Multiple Access (CDMA) network GSM/EDGE and UMTS/HSPA network technologies, Long Term Evolution (LTE), 5G (5th generation mobile networks or 5th generation wireless systems), WiMAX, HSPA+, W-CDMA (Wideband Code-Division Multiple Access), CDMA2000 (also known as C2K or IMT Multi-Carrier (IMT-MC)), Wireless Fidelity (Wi-Fi), Bluetooth, a communication channel as defined herein, or the like, and/or a combination of two or more thereof, that may utilize the teachings of the present application to allow an alert and response integration system device to connect to a wireless network to send and receive data.

Reference in the specification to a user (user 1) is meant to apply to any individual or pet that may be monitored by an alert and response integration system including children, elders, individuals having limited capacity, pets, individuals of concern, and the like. For example, elders may include parents having limited capacity due to age, Alzheimer's, dementia, and the like. Individuals having limited capacity may be those individuals that have a general or specific intellectual and/or physical disability. Pets may include dogs, cats, pigs, other pet types, and the like. Individuals of concern may be individuals awaiting trial, individuals having a prior criminal conviction, an individual demonstrating suspicious behavior at an airport, and the like.

Reference in this specification to a caregiver (caregiver 3) is meant to apply to any individual that may be monitoring a user being monitored by the alert and response integration system including parents, guardians, family members, caregivers, physicians, nurses, schoolteachers, adult children, law enforcement officers, Transportation Security Administration (TSA) officers, and the like.

An administrator (administrator 2) is meant to apply to any entity or individual that is overseeing the caregiver including a law enforcement dispatcher, a law enforcement supervisor, adult children, a hospital administrator, a school administrator, a parent, a third-party service provider, and the like. In one aspect, the user 1 is a school student, the caregiver 3 is a schoolteacher, and the administrator 2 is a principal. In one aspect, the user 1 is a school student, the caregiver 3 is a schoolteacher, and the administrator 2 is a third-party service provider. In one aspect, the user 1 is an elder, the caregiver 3 is an adult child, and the administrator 2 is a third-party service provider. In one aspect, the user 1 is a pet, the caregiver 3 and the administrator 2 is a pet owner. In one aspect, the user 1 is a pet, the caregiver 3 is a pet owner, and the administrator 2 is a third-party service provider.

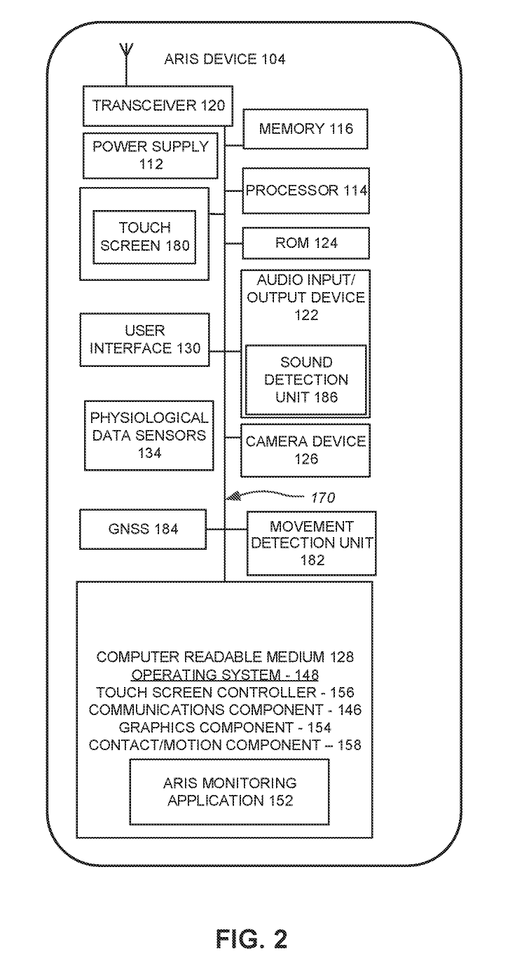

In some aspects, the system may monitor a user 1 by both the administrator 2 and the caregiver 3. In some aspects, the system may monitor a user 1 by only the caregiver 3. In some aspects, the system may monitor a user 1 by only the administrator 2. In some aspects, the ARIS device may be provided to each of the user 1, the administrator 2, and the caregiver 3. In this regard, the ARIS device may be implemented utilizing one or more of the components illustrated in FIG. 2. In one aspect, the ARIS device may be implemented utilizing one or more of the components illustrated in FIG. 2 and programmed for specific use for each of the user 1, the administrator 2, and the caregiver 3. In other words, the ARIS device may utilize a common platform but may be programmed for specific use by one of the user 1, the administrator 2, and the caregiver 3.

Reference in this specification to “one aspect,” “an aspect,” “other aspects,” “one or more aspects” or the like means that a particular feature, structure, or characteristic described in connection with the aspect is included in at least one aspect of the disclosure. The appearances of, for example, the phrase “in one aspect” in various places in the specification are not necessarily all referring to the same aspect, nor are separate or alternative aspects mutually exclusive of other aspects. Moreover, various features are described which may be exhibited by some aspects and not by others. Similarly, various requirements are described which may be requirements for some aspects but not other aspects.

In one exemplary introductory aspect, the disclosure sets forth an alert and response integration system that allows an individual (user 1) to wear a “carrying device” implementation of the ARIS device that may transmit data to a “receiving device” implementation of the ARIS device when the individual (user 1) wearing the carrying device experiences unusual activity (as defined herein) such as takes certain actions and/or has changes in their physiological condition, such as a change in heart rate.

Once a transmission is sent to the “Receiving Device,” the Receiving Device may generate a tone, vibrate, or the like and transmit certain data to an “Alert Device” associated with an administrator 2 and/or caregiver 3. The alert device can transmit data back to the receiving device to capture inquiry, reporting data, and the like and allow sending of an alert to law enforcement if a Carrying Device wearer (user 1) is in danger or is suffering from a severe health issue.

When the “alert device” receives a transmission from the receiving device, the person wearing the “alert device” (administrator 2 and/or caregiver 3) may hear the tone, see the data, and take a specification. In certain aspects, the ARIS device may have human and animal applications. In certain aspects, the ARIS device may include location determination functionality, such as GPS functionality, to allow the wearer to be tracked. In certain aspects, the ARIS device may include transmissions that are data and time stamped. In certain aspects, any data generated, received, and/or transmitted by the ARIS device may be time stamped. In certain aspects, the ARIS device may detect physiological data of the user 1, such as heartrate, stress level, and the like. In certain aspects, the ARIS device data may be captured for customer and law enforcement inquiry and reporting.

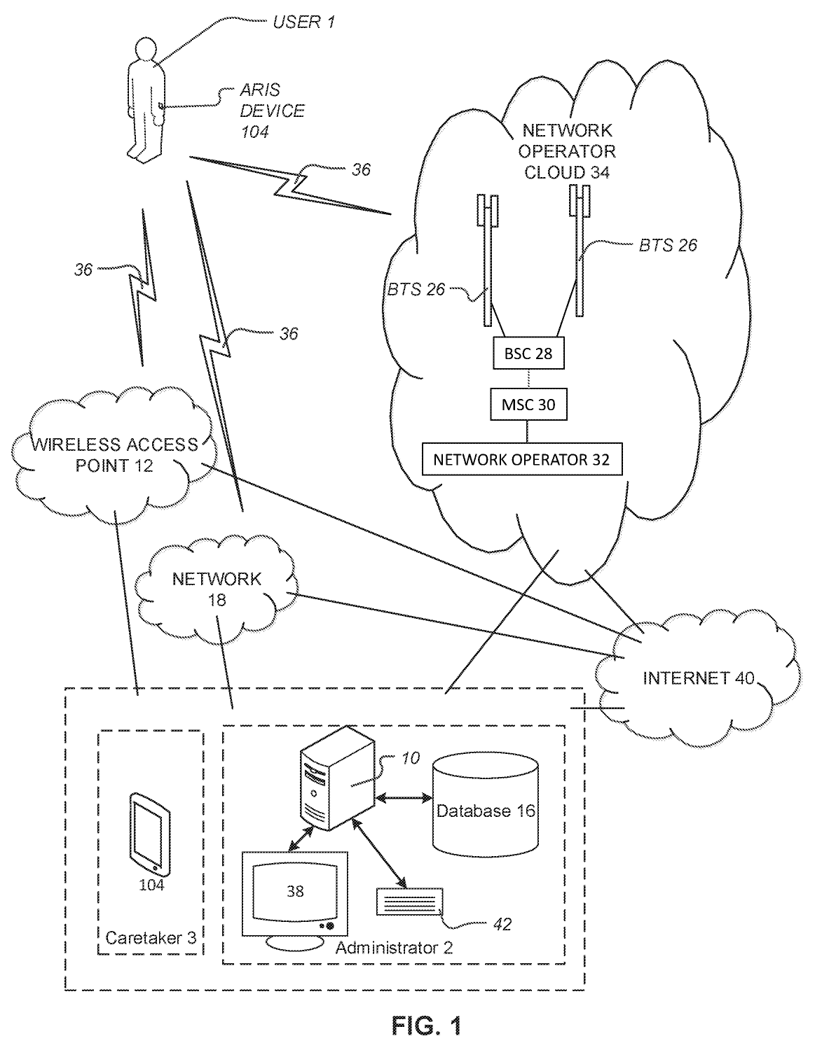

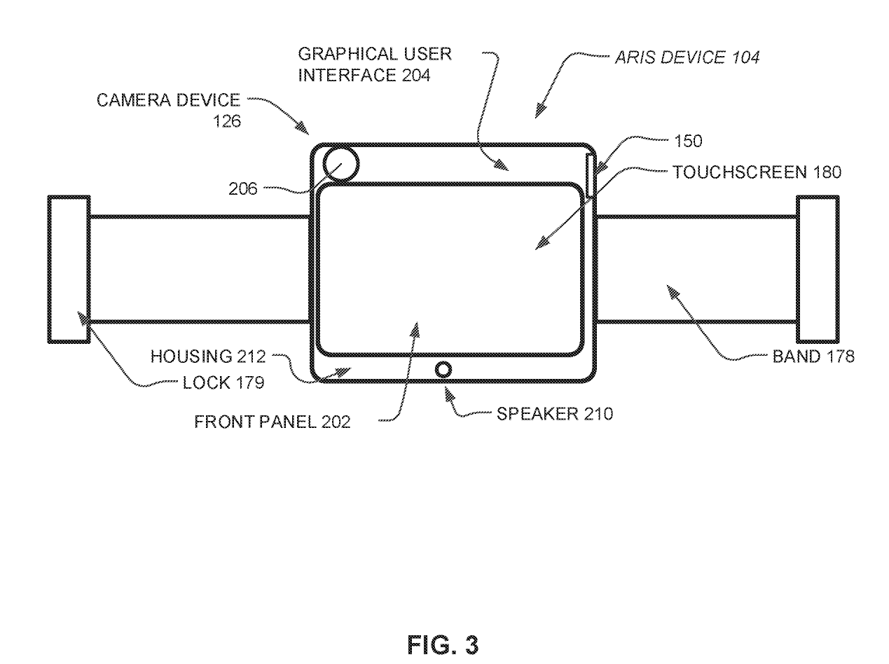

FIG. 1 illustrates an exemplary alert and response integration system device with associated components, in accordance with aspects of the present disclosure. In particular, FIG. 1 shows an alert and response integration system device 104 (ARIS device 104) that may be attached, held and/or carried by the user 1, such as a child, elder, individual having limited capacity, pet, individual of concern, and the like. In one aspect, the ARIS device 104 may include structure to be affixed to the user 1. In one aspect, the ARIS device 104 may include an arm band, wristband, or the like that lockingly attaches the ARIS device 104 to the arm of the user 1. In other aspects, the ARIS device 104 may include structure that attaches the ARIS device 104 to any physical portion of the user 1. In other aspects, the ARIS device 104 may include structure that attaches the ARIS device 104 to the clothing of the user 1 such as spring-loaded safety pin structured device. In other aspects directed to pets, the ARIS device 104 may include structure that attaches the ARIS device 104 to a pet collar of the user 1 that is a pet. In one aspect, the structure may include a band (178 illustrated in FIG. 3) that securely attaches to the ARIS device 104 and encircles a physical portion of the user 1. In one aspect, the structure that securely attaches the ARIS device 104 to the user 1 may be configured to only be removed by the caregiver 3.

The structure that attaches the ARIS device 104 to the user 1 may include a lock structure (179 illustrated in FIG. 3) or other feature that prevents the user 1 from easily removing the ARIS device 104. Moreover, the structure that attaches the ARIS device 104 to the user 1 may include a lock structure or other feature that prevents others from easily removing the ARIS device 104. In one aspect, the structure may include a lock that secures the ARIS device 104 to the user 1 and requires a key to unlock the ARIS device 104, requires an electronic code to unlock the structure, requires an electronic signal to unlock the ARIS device 104, and the like. The lock may be a mechanical or electronic fastening device that is released by a key, keycard, fingerprint, RFID card, security token, keycode, password, or the like, or a combination thereof. In one aspect, the locking feature may be implemented with the band that securely attaches to the ARIS device 104 and encircles a physical portion of the user 1.

The ARIS device 104 may further include a sensor or sensing device configured to determine if the lock is tampered with, damaged, removed, and the like in an unauthorized manner. In one aspect, the ARIS device 104 may include a band having a first locking feature on one end thereof received by corresponding second locking feature on the ARIS device 104. When the first locking feature is removed from the second locking feature, the ARIS device 104 may determine and signal an unauthorized unlocking. In one aspect, the ARIS device 104 may include a band having a wire embedded therein that completes a circuit when the ARIS device 104 is attached to a user. When the wire is broken (circuit is rendered incomplete), the ARIS device 104 may determine and signal an unauthorized unlocking. Accordingly, the above-noted components help to provide a high level of assurance that the ARIS device 104 is securely attached to a user and remains attached to the user. Moreover, should the ARIS device 104 be removed, the caregiver 3 and/or administrator 2 will be informed via the above-noted signaling.

The ARIS device 104 may be configured to capture data, status, location, alerts, other signaling, images, Push-to-talk over cellular (PoC) voice sound, and/or video (hereinafter “user information”) and transmit the user information over a communication channel 36, as defined herein, to the caregiver 3 and/or administrator 2. In one aspect, the user information is transmitted to the caregiver 3 over a communication channel 36 to the ARIS device 104 associated with the caregiver 3. In one aspect, the user information is transmitted to the administrator 2 over a communication channel 36 to the ARIS device 104 associated with the administrator 2. In some aspects, the user information may be encrypted to ensure compliance with the Health Insurance Portability and Accountability Act of 1996 (HIPAA; Pub.L. 104-191, 110 Stat. 1936, enacted Aug. 21, 1996).

In another aspect, the administrator 2 may be associated with an administrator computer 10. In one aspect, the administrator computer 10 may be implemented by a server. The administrator computer 10 may include a Data Capture and Reporting System (DCRS). The DCRS may capture all data associated with the sale, distribution, assignment, activation and monitoring of each ARIS device 104. The identity of each ARIS device 104 wearer may be uniquely identified for tracking and monitoring purposes and for reporting and auditing purposes. This may be especially important if law enforcement personnel are called to a location to search for and/or rescue a person in distress or danger. Customers may be able to inquire on all transactions, alerts, device wearers, dates and times, associated with their ARIS portfolio. Customers will be able to generate pre-defined and custom reports to determine usage, activity, costs, and the like.

The network may include the network operator cloud 34, the Internet 40, a network associated with a wireless access point 12 and/or other networks 18. Only one network is necessary for operation of the ARIS device 104. However, multiple networks are contemplated as well to provide improved coverage. The network operator cloud 34 may include a base transceiver station 26 (BTS), a base station controller 28 (BSC), and a mobile switching center 30 (MSC) overseen by a network operator 32. Other types of wireless networks 12, 18 utilizing a communication channel as defined herein are contemplated as well. The network operator cloud 34 may communicate with the ARIS device 104 over a communication channel 36 as defined herein. The network operator cloud 34 may further communicate over the Internet 40 to the administrator computer 10. The administrator computer 10 may further communicate over the Internet 40 and/or the network operator cloud 34 to the ARIS device 104. In one aspect, the administrator computer 10 may implement web-based communications and commands. The use of the network operator cloud 34 may be beneficial to the user 1 as there are few geographical limitations. Anywhere the user 1 goes, there is likely access to the network operator cloud 34 to send user information.

The administrator 2 may access the administrator computer 10 and a database 16. The administrator computer 10 may be configured to receive user information over the Internet 40, directly from the network operator cloud 34, from the wireless access point 12, and/or another network 18 via a communication channel 36 as defined herein. The administrator computer 10 may be configured to store the user information in the database 16. The administrator computer 10 may also be configured to display the user information on a display 38. The display 38 may be any type of electronic display configured to display content. The database 16 may further include other information on the user 1 including one or more of user name, user address, caregiver name, caregiver address, caregiver phone number and any other information associated with the user 1 and the caregiver 3.

The administrator computer 10 may be configured to transmit commands to the ARIS device 104 through an input device 42 such as a keyboard. The commands may include initiation of video streaming, the stopping of video streaming, location requests, tracking information requests, messages, text messages, email messages, alerts, requests for user information, Push-to-talk over cellular (PoC) functionality, and the like to the ARIS device 104. The administrator computer 10 may be configured to further receive messages, text messages, email messages, alerts, locations, tracking information, user information, Push-to-talk over cellular (PoC) functionality, and the like from the ARIS device 104. Moreover, the administrator computer 10 and the ARIS device 104 may be configured to provide any type of communication therebetween.

The ARIS device 104 associated with the caregiver 3 may be configured to transmit commands to the ARIS device 104 associated with the user 1. The commands may include initiation of video streaming, the stopping of video streaming, location requests, tracking information requests, messages, text messages, email messages, alerts, requests for user information, Push-to-talk over cellular (PoC) functionality, and the like. The ARIS device 104 associated with the caregiver 3 may be configured to further receive messages, text messages, email messages, alerts, locations, tracking information, user information, Push-to-talk over cellular (PoC) functionality, and the like from the ARIS device 104 associated with the user 1. Moreover, the ARIS device 104 associated with the caregiver 3 and the ARIS device 104 associated with the user 1 may be configured to provide any type of communication therebetween.

FIG. 2 shows the details of an exemplary alert and response integration system device in accordance with aspects of the disclosure. The ARIS device 104 may include a processor 114, a memory 116, a user interface 130, and the like. The processor 114 may be a central processing unit, microprocessor, dedicated hardware, or the like configured to execute instructions including instructions related to software programs. The ARIS device 104 may include a display 118. The display 118 may be a liquid crystal display having a backlight to illuminate the various color liquid crystals to provide a colorful display. The user interface 130 may be any type of physical input having one or more buttons, switches, and the like and/or may be implemented as a touchscreen 180. In one aspect, the ARIS device 104 may be implemented with a wireless phone or the like configured to provide the additional functionality as defined herein.

The ARIS device 104 may further include in the memory 116 or separate from the memory 116, a computer readable memory 128, an operating system 148, a communication component 146, a contact/motion component 158, a touchscreen controller 156, a graphics component 154 and the like. The operating system 148 together with the various components providing software functionality for each of the components of the ARIS device 104. The ARIS device 104 may further include a read-only memory 124 (ROM), and a power supply 112 such as a battery.

The memory 116 may include a high-speed random-access memory. Also, the memory 116 may be a non-volatile memory, such as magnetic fixed disk storage, flash memory, cloud-based storage, or the like. The various components of the ARIS device 104 may be connected through various communication lines including a data bus 170.

The ARIS device 104 may further include one or more physiological data sensors 134. The one or more physiological data sensors 134 may sense physiological data of the user 1. The physiological data may include one or more of a heart rate, pulse oximetry, blood glucose, perspiration rate, water submersion, breathing rate, and the like. The physiological data may be indicative of the well-being, state of mind, or the like of the user 1. For example, the physiological data may be indicative of heightened stress, heightened anxiety, heightened nervousness, possible illness, and the like. In this regard, physiological data indicating heightened stress, heightened anxiety, heightened nervousness, possible illness, and the like may be provided as user information to the administrator 2 and/or caregiver 3 for possible intervention or other action including calling law enforcement, emergency medical services, and the like.

The ARIS device 104 utilized by the caregiver 3 also may include one or more physiological data sensors 134 as described herein. The one or more physiological data sensors 134 may sense physiological data of the caregiver 3 to ensure the well-being of the caregiver 3.

Implementations of the physiological data sensors 134 that measure heart rate may include a conductive smart fabric that operates in conjunction with the processor 114 to analyze an EKG signal to determine heart rate. In other aspects, optics may be utilized to measure heart rate using Infrared light. In this aspect, the physiological data sensor 134 may produce infrared light by an internal light source, as Infrared light is absorbed by the blood, the physiological data sensors 134 measure the amount that the infrared light is darkened. If it is determined to be significantly darker, due to the pulse causing a temporary increase in the amount of blood that is travelling through the measured area, it is counted as a heart pulse. Other types of heart rate monitors are contemplated as well.

Implementations of the physiological data sensors 134 that measure pulse oximetry may include a sensor device that may be placed on the user's body. The physiological data sensors 134 may pass two wavelengths of light through the body part to a photodetector. The physiological data sensors 134 may measure the changing absorbance at each of the wavelengths, allowing it to determine the absorbances due to the pulsing arterial blood alone, excluding venous blood, skin, bone, muscle, fat, and the like. In other aspects, the physiological data sensors 134 may utilize reflectance pulse oximetry. Other types of pulse oximetry monitors are contemplated as well.

Implementations of the physiological data sensors 134 that measure blood glucose may include a continuous glucose monitor (CGM) that determines glucose levels on a continuous basis that may include a disposable glucose sensor placed just under the skin. Additionally, other non-invasive technologies may be utilized that include near IR detection, ultrasound, and dielectric spectroscopy. Other types of glucose monitors are contemplated as well.

Implementations of the physiological data sensors 134 that measure perspiration rate may include measuring electrodermal activity, skin conductance, galvanic skin response (GSR), electrodermal response (EDR), psychogalvanic reflex (PGR), skin conductance response (SCR), sympathetic skin response (SSR) and skin conductance level (SCL). In this regard, the physiological data sensor 134 may include a sensor to sense a perspiration rate that may be implemented, for example, by galvanic skin response sensor. Other types of perspiration rate monitors are contemplated as well. Moreover, the physiological data sensors 134 that measure perspiration rate may include additional functionality to determine if the ARIS device 104 is submerged in water. This aspect has applicability in preventing accidental drowning by the user 1.

Implementations of the physiological data sensors 134 that measure respiratory rate may include impedance pneumography, capnography, electrocardiogram, photoplethysmogram, and accelerometry signals utilizing physiological sensors to capture data from the same. Other types of respiratory rate monitors are contemplated as well.

It should be noted however, that the physiological data sensors 134 may be configured to measure any type of physiological data from the user 1. In a further aspect, the physiological data may be indicative of the state of mind, or the like of the user 1. In this aspect, the physiological data such as blood pressure, pulse, respiration, and skin conductivity may provide indicators of deception consistent with a polygraph device. For example, if the user is asked and answers a series of questions that include deceptive answers, this may produce physiological responses that can be differentiated from those associated with non-deceptive answers. These physiological responses may be analyzed by the processor 114 in order to ascertain deception or non-deception and the analysis may be transmitted to the caregiver 3 and/or administrator 2.

Additionally, the ARIS device 104 may include an audio input/output device 122. The audio input/output device 122 may include speakers, speaker outputs, and in the like, providing sound output; and may include microphones, microphone inputs, and the like, for receiving sound inputs. The audio input/output device 122 may include an analog to digital converter and a digital to audio converter for audio input and output functions respectively.

The ARIS device 104 may include a transceiver 120 and the like. The ARIS device 104 may provide radio and signal processing as needed to access a network for services, such as wireless data services, over a communication channel as defined herein. The processor 114 and the transceiver 120 may be configured to process video streaming, call functions, data transfer, Push-to-talk over cellular (PoC) voice sound, and the like and provide other services to the user 1.

The touchscreen 180 of the disclosure may be implemented in the display 118 and may detect a presence and location of a touch of a user within the display area. For example, touching the display 118 of the ARIS device 104 with a finger or hand. The touchscreen 180 may also sense other passive objects, such as a stylus.

In operation, the display 118 may display various objects associated with applications for execution by the processor 114. In this regard, a user may touch the display 118, and in particular the touchscreen 180, to interact with the objects. For example, touching an object may execute an application in the processor 114 associated with the object that is stored in memory 116. Additionally or alternatively, touching an object may open a menu of options to be selected by the user. The display 118 may include a plurality of the objects for the user to interact with. Moreover, the display 118 may include a plurality of screens. The display 118 showing one screen at a time. The user may interact with the display 118 to move a screen into view on the display 118. Various objects may be located in the each of the screens.

The touchscreen 180 may be implemented as a resistive touchscreen, a surface acoustic wave touch screen, a capacitive touch screen, a surface capacitance touchscreen, projected capacitive touch screen, self-capacitance sensors, infrared sensors, dispersive signal technology, acoustic pulse recognition, or the like.

The ARIS device 104 may include a camera device 126. The camera device 126 can include one or more cameras to provide visual input. The ARIS device 104 can include, for example, one camera device 126 on the back side of the ARIS device 104 and another camera device 126 on the front side of the ARIS device 104. The camera device 126 can also capture video in combination with audio from a microphone of the audio input/output device 122. The camera device 126 may include a charge coupled device (CCD), CMOS image sensors, Back Side Illuminated CMOS, or the like. Images captured by the camera device 126 may be converted and stored in various formats including a JPEG file format, RAW feature format such as the Android (operating system) 5.0 Lollipop, and the like. The camera device 126 may include a lens 206 as shown in FIG. 3.

The camera device 126 may operate in conjunction with a monitoring application 152. The monitoring application 152 may be stored in the memory 116, the computer readable memory 128, or the like. The monitoring application 152 may implement full functionality for the camera device 126 to capture images, convert images into a desired format, stream the video content over a communication channel as defined herein, store the images in the memory 116 or the computer readable memory 128, or the like.

In certain aspects, the ARIS device 104 may stream video in real time to the administrator 2 and/or caregiver 3 so that they may take appropriate action based on the content of the video. Moreover, the alert and response integration system in some aspects may be automatic based on environmental sounds and actions sensed by the alert and response integration system. Additionally, the alert and response integration system may be remotely actuated by the administrator 2 and/or caregiver 3, which is beneficial as the user 1 of the alert and response integration system may not always be in a position to initiate video streaming. Moreover, the alert and response integration system of the disclosure may stream video to a remote location which results in a more robust storage of the video content which is not subject to loss if the ARIS device 104 is damaged, stolen, lost, or the like.

In one aspect, the video stream may be compressed using a video codec such as H.264, HEVC, VP8 or VP9. In one aspect, encoded audio and video streams may be assembled in a container bitstream such as MPEG-4, FLV, WebM, ASF or ISMA. The bitstream may be delivered from the ARIS device 104 to the administrator computer 10 using a transport protocol, such as Adobe's RTMP or RTP. In other aspects, technologies such as Apple's HLS, Microsoft's Smooth Streaming, Adobe's HDS and non-proprietary formats such as MPEG-DASH may enable adaptive bitrate streaming over HTTP. A streaming transport protocol may be used to send video to a cloud transcoding service and Content Delivery Network (CDN), which then use HTTP based transport protocols to distribute the video. The streaming client may interact with the streaming server using a control protocol, such as Multimedia Messaging Service (MMS) or Real Time Streaming Protocol (RTSP). Other transport protocols are contemplated as well.

In one aspect, the audio input/output device 122 may include microphones, microphone inputs, and the like, for receiving sound inputs. In this aspect, the audio input/output device 122 in conjunction with the monitoring application 152 may capture sounds and stream the sounds as well to the administrator computer 10 of the administrator 2 and/or the ARIS device 104 of the caregiver 3.

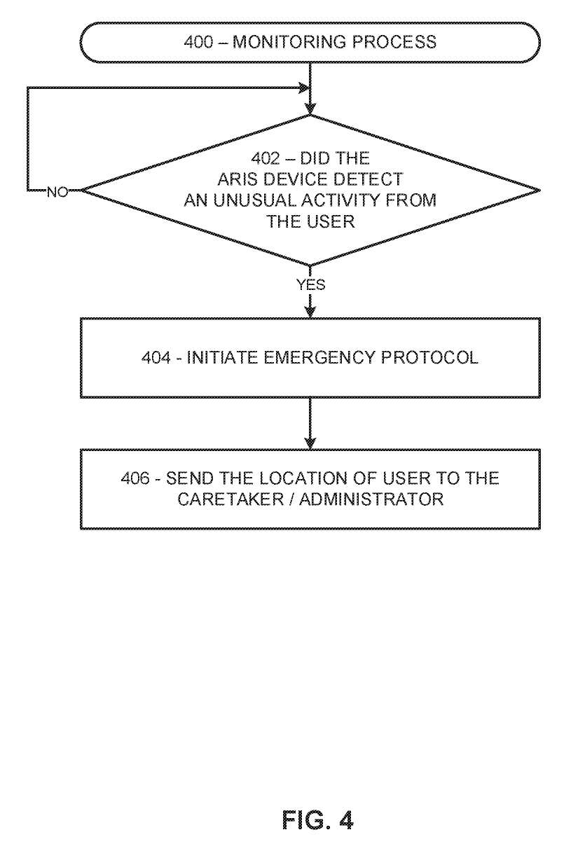

The ARIS device 104 may include a movement detection unit 182. The movement detection unit 182 may include a number of sensors to detect a movement by the user 1. In particular, the movement detection unit 182 may detect a movement indicating unusual or specific movement (unusual activity) by the user 1 suggesting an event that should be subject to monitoring by the ARIS device 104. The movement may include falling, jumping, sudden impact, running, and the like. The movement detection unit 182 may be implemented by any one or more of accelerometers, gyroscopes, altitude sensors, and/or the like. The movement detection unit 182 may further include analog-to-digital converters, filters, and the like to process the signals associated with any of the sensors. In one aspect, the monitoring application 152 may implement an emergency protocol such as video streaming when the unusual or specific movement is detected. The data associated with an unusual movement detected by the movement detection unit 182 may be forwarded to the processor 114 in conjunction with the monitoring application 152. Thereafter, the transceiver 120 may communicate the data associated with unusual movement over a network to the administrator computer 10. The monitor application 152 may implement various aspects of the disclosure including the monitoring process 400 illustrated in FIG. 4.

In one aspect, the audio input/output device 122 may include microphones, microphone inputs, and the like, for receiving sound inputs. In particular, the audio input/output device 122 may detect unusual or specific sounds (unusual activity) by the user 1 suggesting an event that should be subject to monitoring by the ARIS device 104. The unusual or specific sounds may include shouting, screaming, yelling, loud voices, gunshots, and the like. In one aspect, the monitoring application 152 may implement an emergency protocol such as video streaming when the unusual or specific sounds are detected. In one aspect, the audio input/output device 122 may be implemented as a sound detection unit 186.

The computer readable medium 128 may be configured to store the monitoring application 152. For the purposes of this disclosure, the computer readable medium 128 stores computer data, which may include computer program code that may be executable by the processor 114 of the ARIS device 104 in machine readable form. By way of example, and not limitation, the computer readable medium 128 may include computer readable storage media, for example tangible or fixed storage of data, or communication media for transient interpretation of code-containing signals. Computer readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules, or other data. In one or more aspects, the actions and/or events of a method, algorithm, or module may reside as one or any combination or set of codes and/or instructions on a computer readable medium 128 or machine readable medium, which may be incorporated into a computer program product.

According to another aspect of the disclosure, the ARIS device 104 and/or the administrator computer 10 may estimate the location of the ARIS device 104 based, at least in part, on a global navigation satellite system (GNSS 184). In another aspect, a network operator cloud 34 may implement location determination based on a specific cell in which the ARIS device 104 connects. In yet another aspect, a network operator cloud 34 may obtain location determination based on triangulation with respect to a plurality of cells in which the ARIS device 104 receives signals. In yet another aspect, a network operator cloud 34 may obtain location determination based on an inertial guidance system associated with the movement detection unit 182. In this regard, the ARIS device 104 may utilize one or more location determination processes to ensure a greater likelihood of location determination.

The alert and response integration system may include a panic input 150. In one aspect, operating the panic input 150 may implement the monitoring application 152 and the associated camera device 126 to start capturing video and stream the video to the administrator computer 10. The panic input 150 may be a button, switch, touch sensitive area, or the like. In one aspect, the panic input 150 may be a button. In one aspect, the panic input 150 may be a button that is recessed into ARIS device 104 to limit inadvertent pressing. In one aspect, the panic input 150 may operate in conjunction with the monitoring application 152. In one aspect, the panic input 150 may operate in conjunction with the user interface 130. In one aspect, the panic input 150 may be implemented as a button that is responsive to a single click, a double-click, a triple click, an extended hold or pressing and/or the like. In one aspect, the panic input 150 may be associated with a particular application stored on the ARIS device 104 and implemented in conjunction with the monitoring application 152 as described in further detail below. In one aspect, the panic input 150 may be implemented with the audio input/output device 122 that may include microphones, microphone inputs, and the like, for receiving sound inputs. In this aspect, the panic input 150 may be initiated by voice recognition from the user 1. For example, the user 1 may provide a voice command of “help,” for example, that may be interpreted by voice recognition software to implement video streaming. Other types of word based commands are contemplated as well, along with a number of other associated types of functionality.

FIG. 3 shows the details of an exemplary alert and response integration system device in accordance with aspects of the disclosure. In particular, FIG. 3 shows a front panel 202 of the ARIS device 104. The front panel 202 may include the display 118 and a graphical user interface 204 that may implement the touchscreen 180. The front panel 202 may further include the camera device 126 and the lens 206. In one aspect, the lens 206 may be a fisheye lens. In this regard, the fisheye lens may be an ultra wide-angle lens that creates a wide panoramic or hemispherical image in order to capture a better view of the surroundings. In one aspect, the front panel 202 may be a generally flat surface. FIG. 3 further shows the panic input 150 implemented as a button arranged adjacent the front panel 202. FIG. 3 further shows the panic input 150 implemented as a speaker 210 arranged on the front panel 202.

FIG. 3 also illustrates that the ARIS device 104 may include a housing 212. The housing 212 along with other features of the ARIS device 104 may be water resistant, weather resistant, and/or waterproof. In this regard, the housing 212 may be implemented as a waterproof wrapping, a protective case, a nanotechnology coating, and the like. The housing 212 being configured to be water resistant, weather resistant, and/or waterproof may allow the ARIS device 104 to operate in adverse environments (including hot environments and cold environments). For example, the ARIS device 104 may be configured to operate while submerged in water. Moreover, the ARIS device 104 may be able to determine water submersion and alert the caregiver 3, thus prevent accidental drownings.

FIG. 4 shows a process of operating the alert and response integration system device in accordance with aspects of the disclosure. In particular, FIG. 4 shows a monitoring process 400 that may be implemented by the ARIS device 104. In box 402, the ARIS device 104 may determine whether there is an unusual activity associated with the user 1. The unusual activity associated with the user 1 may be a location, a movement, a sound, a panic input, a change in physiological data by the user 1, or visually ascertained and initiated by the caregiver 3 and/or administrator 2, and the like.

For example, the unusual activity may be an unusual movement detected by the movement detection unit 182, as described above. In particular, the movement detection unit 182 may detect a movement indicating unusual or specific movement by the user 1 suggesting an event that should be subject to monitoring by the ARIS device 104. The movement may include falling, jumping, sudden impact, running, and the like. If the movement detection unit 182 detects an unusual movement, the process 400 advances to box 404. Otherwise, if the movement detection unit 182 does not detect an unusual movement, the process 400 continues to monitor activity in box 402.

The unusual activity may be an unusual location detected by the GNSS 184 and/or the movement detection unit 182. For example, the ARIS device 104 of the user 1 may be configured with a safe zone. As long as the ARIS device 104 of the user 1 is located within the safe zone, the location is not considered unusual. When the ARIS device 104 of the user 1 is located outside the safe zone, the location is considered unusual. The safe zone may be a relative distance between the ARIS device 104 of the user 1 and the ARIS device 104 of the caregiver 3. In one aspect, the safe zone may be a predetermined or user-defined distance between the ARIS device 104 of the user 1 and the ARIS device 104 of the caregiver 3. The safe zone may be a school building, school property, a house, a facility, a building, a healthcare facility, a retirement home, a memory center, or the like. In this regard, if the location of the user 1 is unusual, the process 400 advances to box 404. Otherwise, the process 400 continues to monitor activity in box 402.

Alternatively or additionally, box 402 can determine whether there are unusual sounds around the user 1 (unusual activity). In particular, the audio input/output device 122 may detect a sound indicating unusual activity by the user 1 suggesting an event that should be subject to monitoring by the ARIS device 104. The sound may include shouting, yelling, screaming, loud voices, gunshots, and the like. If the audio input/output device 122 detects an unusual sound, the process 400 advances to box 404. Otherwise, if the audio input/output device 122 does not detect an unusual sound, the process 400 continues to monitor activity in box 402.

In box 402, the process may also determine whether the ARIS device 104 has received an alert from the user 1 that is indicative of unusual activity. The alert may be a panic input 150 that may be initiated by a button, switch, touch sensitive area, voice command, or the like as described above. In one aspect the panic input 150 may be a button. In one aspect, the panic input 150 may be implemented with a speaker and the audio input/output device 122 so as to receive voice commands (e.g., a scream for “help”). In one aspect, the panic input 150 may operate in conjunction with the user interface 130. In one aspect, the panic input 150 may be implemented as a button that is responsive to a single click, a double-click, a triple click, an extended hold or pressing and/or the like. In one aspect, the panic input 150 may be associated with a particular application stored on the ARIS device 104 and implemented in conjunction with the monitoring application 152. If the panic input 150 is actuated, the process 400 advances to box 404. Otherwise, if the panic input 150 is not actuated, the process 400 continues to monitor activity in box 402.

The unusual activity may be a change in physiological data detected by the physiological data sensors 134, as described above. In particular, the physiological data sensors 134 may detect an unusual condition of the user 1 suggesting an event that should be subject to monitoring by the ARIS device 104. The physiological data including heart rate, pulse oximetry, blood glucose, perspiration rate, water submersion, breathing rate, and the like. If the physiological data sensors 134 detect a change in physiological condition that exceeds a threshold, the process 400 advances to box 404. Otherwise, the process 400 continues to monitor activity in box 402.

In box 402, the process 400 may determine whether the ARIS device 104 has received an unusual activity alert from the administrator 2 and/or caregiver 3. In this regard, the administrator 2 and/or caregiver 3 may determine that the user 1 requires monitoring for a particular situation. In this regard, the administrator 2 can initiate an unusual activity monitoring request in the administrator computer 10. Likewise, the caregiver 3 can initiate an unusual activity monitoring request with their ARIS device 104. This monitoring request may be sent to the ARIS device 104 over one or more of the Internet 40, network operator cloud 34, wireless access point 12, and/or network 18 on a communication channel as defined herein. If monitoring request has been received, the process 400 advances to box 404. Otherwise, if there is no monitoring request the process 400 advances back to box 402.

In box 404, the ARIS device 104 may initiate an emergency protocol based on the determination of unusual activity associated with box 402. The emergency protocol may include one or more of the following: sending an alert, operating the camera 126 to capture images, operating the camera 126 to capture video, operating the audio input output device 122 to capture audio, contacting the police, contacting emergency medical services (EMS), and the like.

In box 404, the ARIS device 104 may send an alert to the caregiver 3 ARIS device 104, the administrator 2 ARIS device 104, and/or the administrator computer 10. The alert may include the name of the user 1 along with the user information that produced the alert.

In box 404, the ARIS device 104 may initiate operation of the camera device 126 and stream video to the caregiver 3 ARIS device 104, the administrator 2 ARIS device 104, and/or the administrator computer 10. In particular, the monitoring application 152 and the associated camera device 126 may start capturing video and stream the video to the caregiver 3 ARIS device 104, the administrator 2 ARIS device 104, and/or the administrator computer 10 as described above.

In box 404, the ARIS device 104 may initiate operation of the camera device 126 and provide still images to the caregiver 3 ARIS device 104, the administrator 2 ARIS device 104, and/or the administrator computer 10. In one aspect, the monitoring application 152 and the associated camera device 126 may start capturing images and transmit the images to the caregiver 3 ARIS device 104, the administrator 2 ARIS device 104, and/or the administrator computer 10 as described above.

In box 404, the ARIS device 104 may initiate operation of the audio input device 122 and stream audio to the caregiver 3 ARIS device 104, the administrator 2 ARIS device 104, and/or the administrator computer 10. In one aspect, the monitoring application 152 and the associated audio input device 122 may start capturing audio and stream the audio to the caregiver 3 ARIS device 104, the administrator 2 ARIS device 104, and/or the administrator computer 10 as described above.

Finally, in box 406 the ARIS device 104 may send the location of user 1 to the caregiver 3 ARIS device 104, the administrator 2 ARIS device 104, and/or the administrator computer 10. In this regard, the ARIS device 104 and/or the administrator computer 10 may estimate the location of the ARIS device 104 based, at least in part, on a global navigation satellite system (GNSS 184). In another aspect, a network operator cloud 34 may implement location determination based on a specific cell in which the ARIS device 104 connects. In yet another aspect, a network operator cloud 34 may obtain location determination based on triangulation with respect to a plurality of cells in which the ARIS device 104 receives signals. In yet another aspect, a network operator cloud 34 may obtain location determination based on an inertial guidance system associated with the movement detection unit 182. Additionally, the ARIS device 104 may send the location of the user 1 to the caregiver 3 ARIS device 104, the administrator 2 ARIS device 104, and/or the administrator computer 10 at any point in time as requested by the caregiver 3 and/or administrator 2.

In some aspects, the process 400 may utilize artificial intelligence to set and redefine thresholds of unusual activity in order to increase accuracy. Similarly, the physiological data obtained from the physiological data sensors 134 may utilize artificial intelligence to set and redefine thresholds of what is considered physiological data of concern in order to increase accuracy. The artificial intelligence may utilize any number of approaches including one or more of cybernetics and brain simulation, symbolic, cognitive simulation, logic-based, anti-logic, knowledge-based, sub-symbolic, embodied intelligence, computational intelligence and soft computing, machine learning and statistics, and the like.

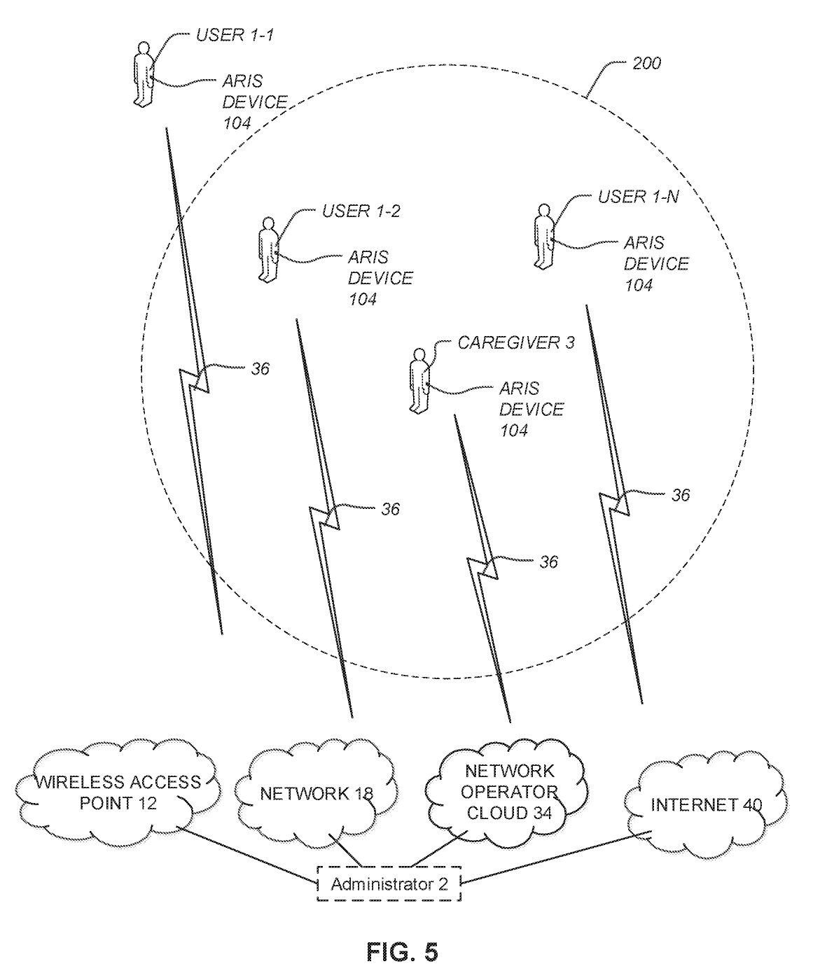

FIG. 5 illustrates an exemplary implementation of an alert and response integration system device with associated components, in accordance with aspects of the present disclosure. In particular, FIG. 5 illustrates a school-based implementation of the ARIS device 104. In this regard, one or more children (users 1-1, 1-2, . . . , 1-N) are each provided with the ARIS device 104. A teacher is assigned as the caregiver 3 and the teacher includes their own ARIS device 104. The ARIS device 104 of the teacher/caregiver 3 may be located within a predefined area 200. The predefined area 200 may be programmed in the ARIS device 104 or factory set in the ARIS device 104. In one aspect, the predefined area 200 may be a predetermined or user-defined distance from the ARIS device 104 of the teacher/caregiver 3. In this regard, if a school class is on a field trip, the predefined area 200 may move with the ARIS device 104 of the teacher/caregiver 3.

The ARIS device 104 for the one or more children (users 1-1, 1-2, . . . , 1-N) may each provide a current location with respect to the teacher/caregiver 3. When one of the children is outside the predefined area 200, such as the child illustrated as the user 1-1, the ARIS device 104 of the user 1-1 may recognize the child's location as unusual activity and alert the teacher/caregiver 3 of this unusual activity consistent with the process 400.

The ARIS device 104 for the one or more children (users 1-1, 1-2, . . . , 1-N) may further recognize other unusual activity and alert the teacher/caregiver 3 of this unusual activity consistent with the process 400. For example, if the physiological data sensor 134 of one of the children is indicating unusual activity through the collection of physiological data.

As a specific example, a parent/teacher would put the ARIS device 104 ARIS (Carrying Device) on their child's wrist. The parent/teacher would put the ARIS device 104 (an ARIS Alert Device) on their own wrist. The parent/teacher would program the allowed distance parameter of a certain number of feet allowable for the child to be separate from the parent/teacher. The Carrying Device may include a location determination device that can prompt the Alert Device of the child's geographic location at any time once activated. The Carrying Device also has the ability to monitor the physiological condition, such as heart rate, of the child with pre-defined levels that prompt alert of severe stress, danger, or death (no heart rate) to the parent or guardian's Alert device. Each Alert Device can be programmed to receive data from any of its pre-defined group of Carrying Devices. A unique identification number may be associated with each Carrying Device and each Alert Device. Therefore, a teacher will know which student is assigned a given Carrying Device. The teacher will also know when a given Carrying Device attached to a student has been activated to begin tracking and monitoring. When the Alert Device is sent information pertaining to a particular Carrying Device, the teacher will see which student has reached the edge of the allowed distance parameter. The teacher may also know which student is in peril because their physiological data, such as heart rate, has risen to an alert level or has stopped.

When the parent, guardian or teacher and the child or children go outside, and any of the pre-defined children reach the edge of the allowed distance parameter, or have a severely elevated or stopped heart rate, the Alert Device of the associated caregiver would prompt the caregiver to quickly find and bring the child back inside the allowed distance parameter and/check to ensure his/her health and safety.

The Carrying Device may contain a panic button that will allow a child to alert their parent, guardian, teacher or caregiver if they are in danger and are seriously hurt or sick. When the panic button is hit, the device will automatically take a picture or video of the surrounding area. If an unauthorized person attempts to remove the carrying device, a picture or video of that person is automatically taken and an alert is automatically sent to the ARIS device 104 associated with their parent, guardian, teacher, administrator, or caregiver.

Aspects of FIG. 5 may be equally applicable to a family situation. In this regard, one or more children (users 1-1, 1-2, . . . , 1-N) are each provided with the ARIS device 104. A parent, grandparent, babysitter and the like is assigned as the caregiver 3 and they have their own ARIS device 104. For example, a child wears the ARIS device 104 (an ARIS carrying device). The parent or guardian wears another ARIS device 104 (an ARIS alert device). When the child reaches the edge of a safe zone, the ARIS carrying device sends a signal to the response device which will put out a tone and will transmit an alert the parent or guardian wearing the alert device that the child has left the safe area. At that time, the child's location can continue to be tracked. The parent or guardian can also be alerted when the child's stress or anxiety level becomes high. When this happens, the parent or guardian will also be able to track the location of the child. The parent or guardian can also be prompted if someone is attempting to remove the device from the child.

This implementation of the ARIS device 104 has a number of benefits. For the child, it gives them a higher probability of staying safe, gives them a higher probability of having their anxiety or severe stress quickly addressed by the parent or guardian, gives them a higher probability of being found quickly if they are taken or wonder off, and the like. For the parent, a parent or guardian will know where the child is and that they are safe, a parent or guardian will know as soon as a child approaches the edge of a secure location or leaves that secure location (predefined area), and a parent or guardian can find a lost or stolen child sooner. Moreover, this lowers stress and anxiety for the parent or guardian, builds trust that a caregiver or family member is not creating undo stress or anxiety for the child (abuse), and the like. In some aspects, all captured data (user information) may be made available for the customer inquiry and reporting, and for law enforcement (as requested). All data may include device identification, all associated transmission and receiving of data and associated date and time.

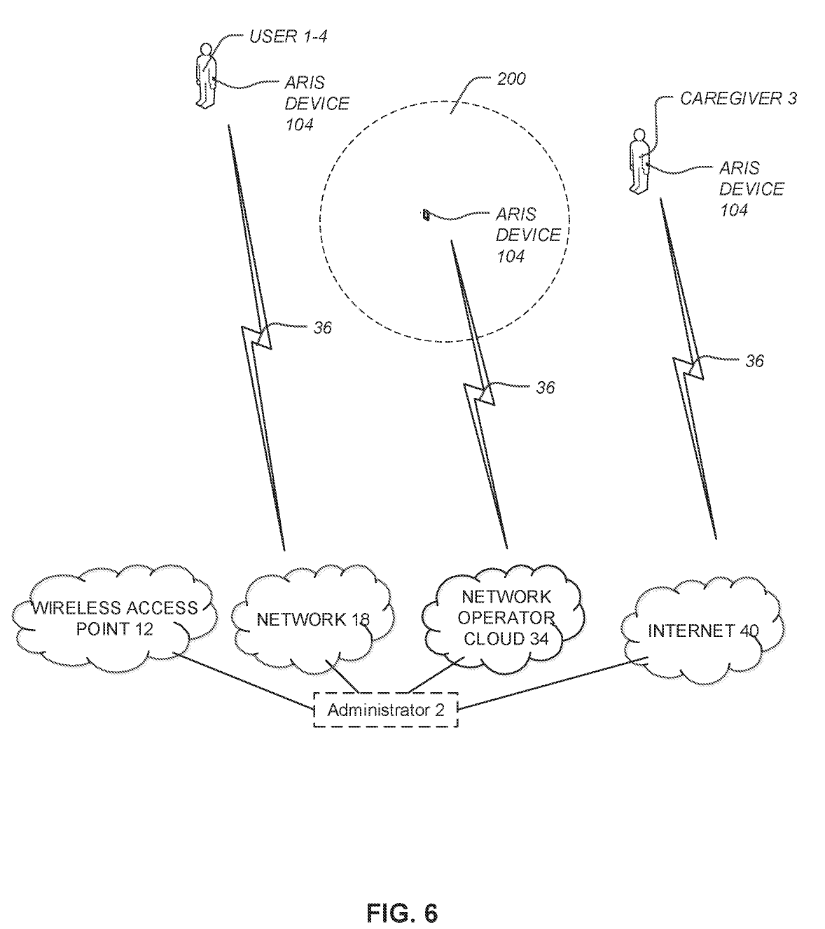

FIG. 6 illustrates an exemplary implementation of an alert and response integration system device with associated components, in accordance with aspects of the present disclosure. In particular, FIG. 6 illustrates a home-based implementation of the ARIS device 104. In this regard, an individual (user 1-4) is provided the ARIS device 104. In this aspect, the individual may be an elder, individual of limited capacity, and individual concern, or the like. The caregiver 3 may be provided with their own ARIS device 104. The ARIS device 104 of the caregiver 3 may not have to be located within a predefined area 200. The predefined area 200 may be programmed in the ARIS device 104 or factory set in the ARIS device 104. In another aspect, a base station implementation of the ARIS device 104 located in the home may provide a location to define the predefined area 200.

The ARIS device 104 for the individual (user 1-4) may provide a current location with respect to the predefined area 200. When individual is outside the predefined area 200, such as the individual illustrated as the user 1-4, the ARIS device 104 of the user 1-4 may recognize the individual's location as unusual activity and alert the caregiver 3 of this unusual activity consistent with the process 400.

The ARIS device 104 for the individual (user 1-4) may further recognize other unusual activity and alert the caregiver 3 of this unusual activity consistent with the process 400. For example, if the physiological data sensor 134 of the individual is indicating unusual activity through the collection of physiological data.

As a particular example, a person with Alzheimer's or dementia wears the ARIS device 104 (an ARIS carrying device). His or her guardian wears another ARIS device 104 (an ARIS alert device). When the adult with Alzheimer's or dementia reaches the edge of a safe zone, the carrying device may send a signal to the response device which may alert the guardian wearing the alert device of the location of the person with Alzheimer's or dementia. The guardian can also be alerted when the person's stress or anxiety level becomes high. When this happens, the guardian will also know the location of the person. The ARIS device 104 of the guardian may also be alerted if the carrying device is removed or forcibly removed.

This implementation has a number of benefits for the person with Alzheimer's or dementia including: provides them with a higher probability of staying safe, gives them a higher probability of having their anxiety or severe stress quickly addressed by the guardian, caregiver or relative, gives them a higher probability of being found quickly if they wander or are taken, and the like. This implementation has a number of benefits for the Guardian or Caregiver including: the guardian or caregiver will know where the person is and that they are safe, the guardian or caregiver will know as soon as the person approaches the edge of a secure location or leaves that secure location (programmed as a predefined area 200), the guardian or caregiver can find a lost or kidnapped person sooner, and the like. Moreover, this implementation lowers stress and anxiety, builds trust that a caregiver or family member is not creating undo stress or anxiety for the person with Alzheimer's or dementia (abuse), and the like. Moreover, this aspect also works well for children or adults with Special Needs, Autism, Asperger's, or other issues.

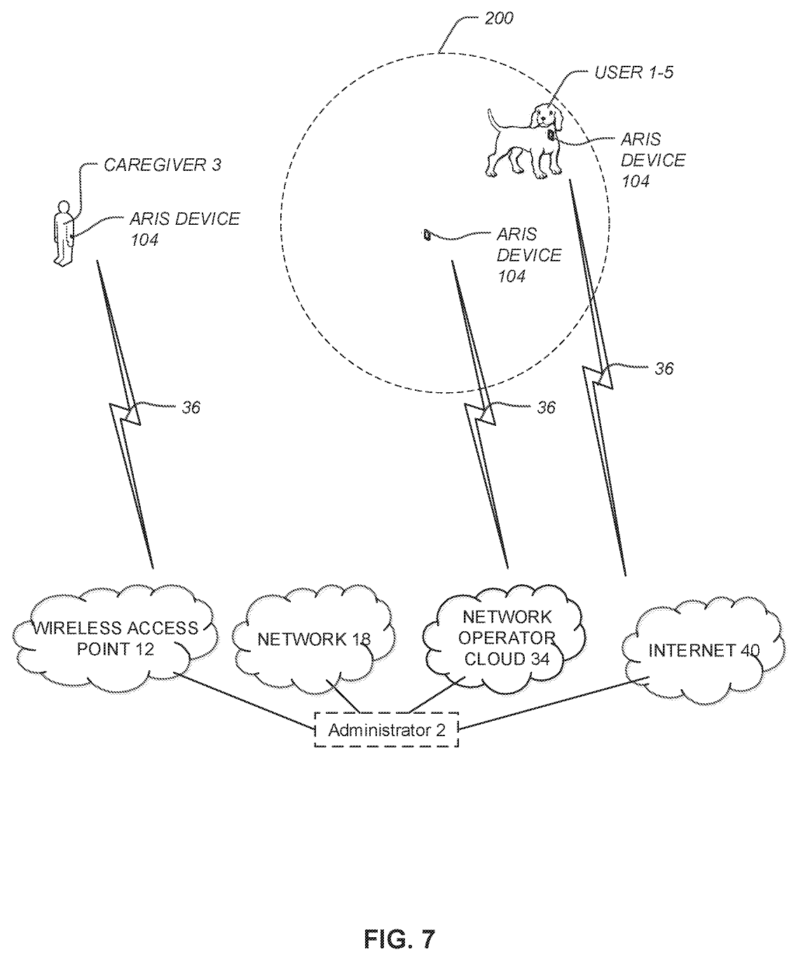

FIG. 7 illustrates an exemplary implementation of an alert and response integration system device with associated components, in accordance with aspects of the present disclosure. In particular, FIG. 7 illustrates a pet-based implementation of the ARIS device 104. In this regard, a pet (user 1-5) is provided the ARIS device 104. The caregiver 3 may be provided with their own ARIS device 104. The ARIS device 104 of the caregiver 3 may not have to be located within a predefined area 200. The predefined area 200 may be programmed in the ARIS device 104 or factory set in the ARIS device 104. In another aspect, a base station implementation of the ARIS device 104 located in the home may provide a location to define the predefined area 200.

The ARIS device 104 for the pet (user 1-5) may provide a current location with respect to the predefined area 200. When the pet is within the predefined area 200, the ARIS device 104 of the user 1-5 may recognize the pet's location as unusual activity and alert the caregiver 3 of this unusual activity consistent with the process 400. In this regard, the predefined area 200 may be a location near a door where the pet may be desiring to be let out. The caregiver 3 will be alerted to this pet's location so that the pet can be let out.

The ARIS device 104 for the pet (user 1-5) may further recognize other unusual activity and alert the caregiver 3 of this unusual activity consistent with the process 400. For example, if the physiological data sensor 134 of the pet is indicating unusual activity through the collection of physiological data.

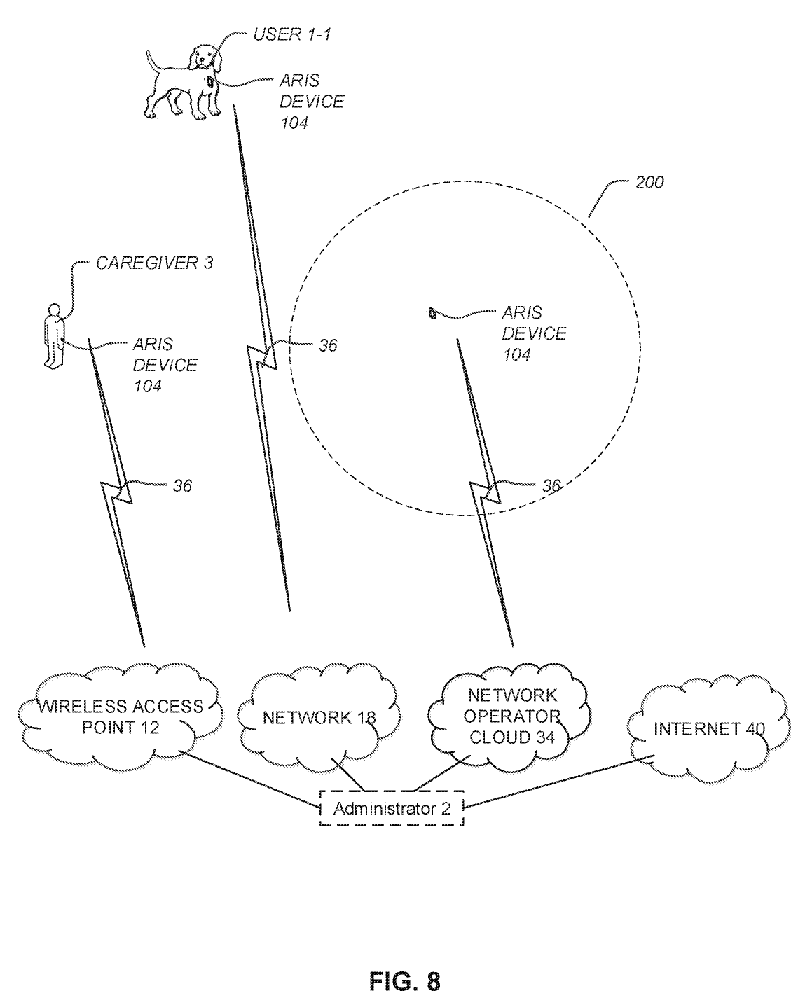

FIG. 8 illustrates an exemplary implementation of an alert and response integration system device with associated components, in accordance with aspects of the present disclosure. In particular, FIG. 8 illustrates another aspect of FIG. 7 focusing on a lost pet scenario. In this regard, the predefined area 200 may be set as a yard in which the pet habitats. If the pet leaves the yard/predefined area 200, then the pet owner or caregiver 3 may be alerted.

As a particular example, a dog wears the ARIS device 104 (an ARIS carrying device). His owner has placed a number of ARIS devices 104 (ARIS response devices) in different rooms throughout the house. When the dog reaches a pre-defined exit door and stays there for a pre-set period of time, the ARIS response device may put out a tone and transmit an alert to the owner wearing another ARIS device 104 (the “alert device”) that the dog is waiting at the front door to go outside.

In this regard, the ARIS device 104 has a number of benefits to the pet owner including: it protects carpets and floors from having to be cleaned and/or replaced, it keeps the house healthy and fresh smelling, it enhances the relationship between the dog and the owner, it lowers the stress of the owner who worries about not being near the front door when the dog alerts that he needs to go out, it can lower veterinarian bills from UTIs and other health issues. Moreover, the ARIS device 104 has a number of benefits to the pet including: it enhances the health of the dog since he can go when he needs to go, it enhances the dog's feeling that his owner is there for him, it reinforces that bladder and bowel relief is done outside, so the dog can be house broken faster, it lowers the stress of the dog, and the like. Moreover, if the pet owner is getting older, this implementation will help the pet owner remember to take the dog out. This scenario will work well for a caregiver since the caregiver will be less aware of the dog's regular pattern of needing to go outside. It will also allow the dog to stay within its normal and learned pattern of when it will be taken out and how to notify someone when it needs to go out.

All captured data may be made available for the customer inquiry and reporting, and for law enforcement (as requested) and for purposes of protecting the third-party service provider. All data may include device identification, all associated transmission and receiving of data and associated date and time.

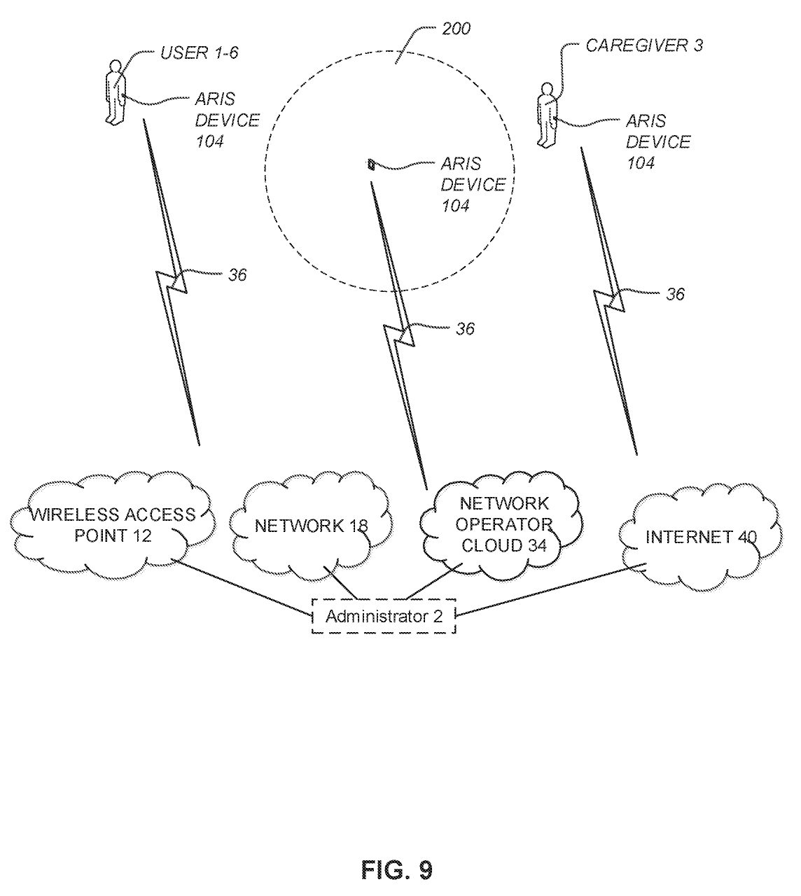

FIG. 9 illustrates an exemplary implementation of an alert and response integration system device with associated components, in accordance with aspects of the present disclosure. In particular, FIG. 9 illustrates a law enforcement-based implementation of the ARIS device 104. In this regard, an individual of concern (user 1-6) is provided the ARIS device 104. The caregiver 3 may be provided with their own ARIS device 104. The ARIS device 104 of the caregiver 3 may not have to be located within a predefined area 200. The predefined area 200 may be programmed in the ARIS device 104 or factory set in the ARIS device 104. In another aspect, a base station implementation of the ARIS device 104 may be located in the home may provide a location to define the predefined area 200.

The ARIS device 104 for the individual (user 1-6) may provide a current location with respect to the predefined area 200. When individual is outside the predefined area 200, such as the individual illustrated as the user 1-6, the ARIS device 104 of the user 1-6 may recognize the individual's location as unusual activity and alert the caregiver 3 of this unusual activity consistent with the process 400.

The ARIS device 104 for the individual (user 1-6) may further recognize other unusual activity and alert the caregiver 3 of this unusual activity consistent with the process 400. For example, if the physiological data sensor 134 of the individual is indicating unusual activity through the collection of physiological data. In this aspect, the ARIS device 104 may be further configured with the polygraph functionality described above in order to ascertain deceptive actions.

In another aspect, an individual that is indicating unusual behavior in a public place may be determined to be an individual of concern. For example, the public place may include airports, federal buildings, public buildings, and the like. A law enforcement officer may request that the individual indicating unusual behavior carry the ARIS device 104. The law enforcement officer would carry their own ARIS device 104 to monitor the individual. The purpose would be to monitor any unusual activity, possible deceptive actions, and the like to ensure that this individual of concern does not act inappropriately. As another example, all individuals in a particular public place such as an airport may be issued the ARIS device 104. For example, ticketed passengers making their way through an airport terminal including the airport security, may be issued the ARIS device 104. The ARIS device 104 may monitor and report to the TSA any unusual activity (such as elevated heart rate, excessive perspiration, etc.), possible deceptive actions, and the like to ensure that ticketed passengers do not act inappropriately. It is contemplated by the disclosure that this concept may be extended to any other large crowd area, public place, public facility, and the like that requires a higher level of security and oversight.

All captured data may be made available for the customer inquiry and reporting, and for law enforcement (as requested) and for purposes of protecting the third-party service provider. All data may include device identification, all associated transmission and receiving of data and associated date and time.

Accordingly, the disclosure has described an alert and response integration system, device, and process that provides greater capability, usefulness, and the like to protect, benefit, and ensure the well-being, supervision, and/or oversight of children, adults, pets, and/or society as a whole. There are numerous applications for this integrated system. The ARIS device can be used to identify, track and report on the location and stress level of people or animals in any of a number of scenarios. For example, a child at daycare or school, an elderly person at home alone, or a pet being cared for by a new neighbor. The list goes on and on. The ARIS device can also be implemented to prompt the receiving device the second a heartbeat has stopped. This could save a child from SIDS, drowning or suffocation. Because it could detect the ceasing of a heartbeat, it may also detect a heart attack, a hemorrhagic stroke, a sudden death, or the like of an individual such as an elderly person.

In certain aspects, a third-party monitoring service may be associated with the administrator 2. In this regard, the administrator may charge a fee (e.g. monthly fee) for continuing service, provisioning the device, monitoring, reporting, and the like. This allows the administrator 2 to continue to bring in revenue while building long-term customer relationships. This charge may be per device, or in cases of larger numbers of devices, there could be a monthly charge for a set of devices.

For the purposes of this disclosure a computer readable medium stores computer data, which data can include computer program code that is executable by a processor of the SIM or mobile device, in machine readable form? By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and nonremovable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage media, magnetic cassettes, magnetic tape, magnetic disk storage or other magnetic storage devices, or any other physical or material medium which can be used to tangibly store the desired information or data or instructions and which can be accessed by a processor or computing device. In one or more aspects, the actions and/or events of a method, algorithm or module may reside as one or any combination or set of codes and/or instructions on a computer readable medium or machine readable medium, which may be incorporated into a computer program product.

Aspects of the disclosure may include communication channels that may be any type of wired or wireless electronic communications network, such as, e.g., a wired/wireless local area network (LAN), a wired/wireless personal area network (PAN), a wired/wireless home area network (HAN), a wired/wireless wide area network (WAN), a campus network, a metropolitan network, an enterprise private network, a virtual private network (VPN), an internetwork, a backbone network (BBN), a global area network (GAN), the Internet, an intranet, an extranet, an overlay network, Near field communication (NFC), a cellular telephone network, a Personal Communications Service (PCS), using known protocols such as the Global System for Mobile Communications (GSM), CDMA (Code-Division Multiple Access), GSM/EDGE and UMTS/HSPA network technologies, Long Term Evolution (LTE), 5G (5th generation mobile networks or 5th generation wireless systems), WiMAX, HSPA+, W-CDMA (Wideband Code-Division Multiple Access), CDMA2000 (also known as C2K or IMT Multi-Carrier (IMT-MC)), Wireless Fidelity (Wi-Fi), Bluetooth, and/or the like, and/or a combination of two or more thereof. The NFC standards cover communications protocols and data exchange formats, and are based on existing radio-frequency identification (RFID) standards including ISO/IEC 14443 and FeliCa. The standards include ISO/IEC 18092[3] and those defined by the NFC Forum.

According to an example, the global navigation satellite system (GNSS) may include a device and/or system that may estimate its location based, at least in part, on signals received from space vehicles (SVs). In particular, such a device and/or system may obtain “pseudorange” measurements including approximations of distances between associated SVs and a navigation satellite receiver. In a particular example, such a pseudorange may be determined at a receiver that is capable of processing signals from one or more SVs as part of a Satellite Positioning System (SPS). Such an SPS may comprise, for example, a Global Positioning System (GPS), Galileo, Glonass, to name a few, or any SPS developed in the future. To determine its location, a satellite navigation receiver may obtain pseudorange measurements to three or more satellites as well as their positions at time of transmitting. Knowing the SV orbital parameters, these positions can be calculated for any point in time. A pseudorange measurement may then be determined based, at least in part, on the time a signal travels from an SV to the receiver, multiplied by the speed of light. While techniques described herein may be provided as implementations of location determination in GPS and/or Galileo types of SPS as specific illustrations according to particular examples, it should be understood that these techniques may also apply to other types of SPS, and that claimed subject matter is not limited in this respect.

In an aspect, the disclosure may be implemented in any type of mobile smartphones that are operated by any type of advanced mobile data processing and communication operating system, such as, e.g., an Apple iOS operating system, a Google Android operating system, a RIM Blackberry operating system, a Nokia Symbian operating system, a Microsoft Windows Mobile operating system, a Microsoft Windows Phone operating system, a Linux operating system or the like.

The term data as utilized herein includes mobile broadband or wireless Internet access delivered through mobile phone towers over a communication channel as defined herein to computers, mobile phones, wireless devices, and other digital devices as defined herein using portable modems.

Additionally, the various aspects of the disclosure may be implemented in a non-generic computer implementation. Moreover, the various aspects of the disclosure set forth herein improve the functioning of the system as is apparent from the disclosure hereof. Furthermore, the various aspects of the disclosure involve computer hardware that it specifically programmed to solve the complex problem addressed by the disclosure. Accordingly, the various aspects of the disclosure improve the functioning of the system overall in its specific implementation to perform the process set forth by the disclosure and as defined by the claims.

Further in accordance with various aspects of the disclosure, the methods described herein are intended for operation with dedicated hardware implementations including, but not limited to, microprocessors, PCs, PDAs, SIM cards, semiconductors, application specific integrated circuits (ASIC), programmable logic arrays, cloud computing devices, and other hardware devices constructed to implement the methods described herein.

Aspects of the disclosure may be web-based. For example, a server may operate a web application in conjunction with a database. The web application may be hosted in a browser-controlled environment (e.g., a Java applet and/or the like), coded in a browser-supported language (e.g., JavaScript combined with a browser-rendered markup language (e.g., Hyper Text Markup Language (HTML) and/or the like)) and/or the like such that any computer running a common web browser (e.g., Internet Explorer™, Firefox™, Chrome™, Safari™ or the like) may render the application executable. A web-based service may be more beneficial due to the ubiquity of web browsers and the convenience of using a web browser as a client (i.e., thin client). Further, with inherent support for cross-platform compatibility, the web application may be maintained and updated without distributing and installing software on each.

The monitoring application 152 described in the disclosure may be implemented to execute on an Apple™ iOS™ operating system, a Google™ Android™ operating system, a RIM™ Blackberry™ operating system, a Nokia™ Symbian™ operating system, a Microsoft™ Windows Mobile™ operating system, a Microsoft™ Windows Phone™ operating system, a Linux™ operating system or the like. The application may be displayed as an icon. The application may have been downloaded from the Internet, pre-installed, or the like. In some aspects, the application may be obtained from Google PIay™, Android Market™, Apple Store™, or the like digital distribution source. The application may be written in conjunction with the software developers kit (SDK) associated with an Apple™ iOS™ operating system, a Google™ Android™ operating system, a RIM™ Blackberry™ operating system, a Nokia™ Symbian™ operating system, a Microsoft™ Windows Mobile™ operating system, a Microsoft™ Windows Phone™ operating system, a Linux™ operating system or the like.

Aspects of the disclosure may include a server executing an instance of an application or software configured to accept requests from a client and giving responses accordingly. The server may run on any computer including dedicated computers. The computer may include at least one processing element, typically a central processing unit (CPU), and some form of memory. The processing element may carry out arithmetic and logic operations, and a sequencing and control unit may change the order of operations in response to stored information. The server may include peripheral devices that may allow information to be retrieved from an external source, and the result of operations saved and retrieved. The server may operate within a client-server architecture. The server may perform some tasks on behalf of clients. The clients may connect to the server through the network on a communication channel as defined herein. The server may use memory with error detection and correction, redundant disks, redundant power supplies and so on.

Voice recognition software may be utilized in various aspects of the systems and methods. Users may be able to vocalize, rather than utilizing other input processes. For example, the voice recognition software may be configured for generating text from voice input from a microphone or other voice input. A speech signal processor may convert speech signals into digital data that can be processed by the processor. The processor may perform several distinct functions, including serving as the speech event analyzer, the dictation event subsystem, the text event subsystem, and the executor of the application program. The speech signal processor may generate speech event data and transmit this data to the processor to be processed first by the speech event analyzer. The speech event analyzer may generate a list or set of possible candidates among the system recordings that represent or match the voice input processed by the speech signal processor. The speech event analyzer may transmit the candidate sets to a dictation event subsystem. The dictation event subsystem may analyze the candidate sets and choose the best match candidate with the highest degree of similarity. This candidate is then considered the correct translation, and the dictation event subsystem forwards the translation to the text event subsystem which in turn inputs the translated text into the device.

While the system and method have been described in terms of what are presently considered to be specific aspects, the disclosure need not be limited to the disclosed aspects. It is intended to cover various modifications and similar arrangements included within the spirit and scope of the claims, the scope of which should be accorded the broadest interpretation so as to encompass all such modifications and similar structures. The present disclosure includes any and all aspects of the following claims.

1. An alert and response integration system device comprising:
at least one physiological data sensor configured to sense physiological data of a user;
at least one location determination device configured to determine a location of the user;
a memory configured to store a monitoring application;
a processor configured to execute the monitoring application and obtain the physiological data from the at least one physiological data sensor and obtain the location of the user from the at least one location determination device;
the processor further configured to generate an alert if the physiological data exceeds a predetermined threshold;
the processor further configured to generate the alert if the location of the user is a particular location;
a transceiver configured to transmit the alert over a wireless network to a monitoring device;
a housing configured to house the at least one physiological data sensor, the at least one location determination device, the memory, the processor, and the transceiver; and
an attachment mechanism associated with the housing, the attachment mechanism being configured to securely affix the housing to the user with a locking mechanism,
wherein the locking mechanism is configured to be unlocked by a caregiver.

2. The alert and response integration system device according to claim 1 wherein the transceiver is further configured to transmit the location and the physiological data of the user over the wireless network to the monitoring device in response to the alert or a monitoring request.

3. The alert and response integration system device according to claim 1 further comprising:
a camera device configured to capture an image;
the processor further configured to operate the camera device to capture the image in response to the alert; and
the transceiver further configured to transmit the image over the wireless network to the monitoring device,
wherein the image comprises at least one of the following: a video image and a still image.

4. The alert and response integration system device according to claim 1 wherein the at least one physiological data sensor comprises a sensor configured to sense at least one of the following: heart rate, pulse oximetry, blood glucose, perspiration rate, water submersion, and breathing rate.

5. The alert and response integration system device according to claim 1 further comprising:
a movement detection unit configured to detect a movement of the housing, the movement detection unit and the processor configured to generate the alert if the movement comprises a particular movement,
wherein the particular movement comprises at least one of the following: falling, jumping, sudden impact, or running.

6. The alert and response integration system device according to claim 1 further comprising:
a sound detection unit configured to detect sounds, the sound detection unit and the processor further configured to generate the alert if the sounds comprise a particular sound,
wherein the particular sound comprises at least one of the following: shouting, screaming, yelling, loud voices, or gunshots.

7. The alert and response integration system device according to claim 1 further comprising:
a panic input configured to receive a panic input from the user; and
the transceiver further configured to transmit the alert generated by the panic input over the wireless network to the monitoring device.

8. The alert and response integration system according to claim 1 wherein the alert and response integration system comprises at least one of the following: a wireless phone, a mobile phone, user equipment, a tablet computer, and a smartphone.

9. A system comprising the alert and response integration system device according to claim 1 and the monitoring device, the monitoring device comprising:
a transceiver configured to receive the alert over the wireless network;
a display configured to display the alert and user information associated with the user; and
an audio output device configured to generate a sound in response to receiving the alert.

10. A system comprising the alert and response integration system according to claim 1 and the monitoring device, the monitoring device comprising:
a computer configured to receive the alert over the wireless network;
a display configured to display the alert and user information associated with the user; and
a database configured to store the alert and the user information.

11. An alert and response integration process comprising:
sensing physiological data of a user with at least one physiological data sensor;
determining a location of the user with at least one location determination device;
storing a monitoring application in a memory;
executing the monitoring application with a processor and the processor obtaining the physiological data from the at least one physiological data sensor and the processor obtaining the location of the user from the at least one location determination device;
generating with the processor an alert if the physiological data exceeds a predetermined threshold;
generating with the processor the alert if the location of the user is a particular location;
transmitting the alert over a wireless network with a transceiver to a monitoring device;
providing a housing configured to house the at least one physiological data sensor, the at least one location determination device, the memory, the processor, and the transceiver; and
providing an attachment mechanism associated with the housing, the attachment mechanism being configured to securely affix the housing to the user with a locking mechanism,
wherein the locking mechanism is configured to be unlocked by a caregiver.

12. The alert and response integration process according to claim 11 further comprising transmitting the location and the physiological data of the user with the transceiver over the wireless network to the monitoring device in response to the alert or a monitoring request.

13. The alert and response integration process according to claim 11 further comprising:
providing a camera device configured to capture an image;
operating the camera device in response to the processor to capture the image in response to the alert; and
transmitting the image over the wireless network with the transceiver to the monitoring device,
wherein the image comprises at least one of the following: a video image and a still image.

14. The alert and response integration process according to claim 11 wherein the at least one physiological data sensor comprises a sensor configured to sense at least one of the following: heart rate, pulse oximetry, blood glucose, perspiration rate, water submersion, and breathing rate.

15. The alert and response integration process according to claim 11 further comprising:
detecting a movement of the housing with a movement detection unit, the movement detection unit and the processor configured to generate the alert if the movement comprises a particular movement,
wherein the particular movement comprises at least one of the following: falling, jumping, sudden impact, or running.

16. The alert and response integration process according to claim 11 further comprising:
detecting sounds with a sound detection unit, the sound detection unit and the processor configured to generate the alert if the sounds comprise a particular sound,
wherein the particular sound comprises at least one of the following: shouting, yelling, loud voices, screaming, or gunshots.

17. The alert and response integration process according to claim 11 further comprising:
receiving a panic input from the user with a panic input; and
transmitting with the transceiver the alert generated by the panic input over the wireless network to the monitoring device.

18. The alert and response integration process according to claim 11 wherein the alert and response integration process is provided in at least one of the following: a wireless phone, a mobile phone, user equipment, a tablet computer, and a smartphone.

19. The alert and response integration process according to claim 11 further comprising providing a system comprising the monitoring device, the monitoring device comprising:
a transceiver configured to receive the alert over the wireless network;
a display configured to display the alert and user information associated with the user; and
an audio output device configured to generate a sound in response to receiving the alert.

20. The alert and response integration process according to claim 11 further comprising providing a system comprising the monitoring device, the monitoring device comprising:
a computer configured to receive the alert over the wireless network;
a display configured to display the alert and user information associated with the user; and
a database configured to store the alert and the user information.
